(19) United States
(12) Patent Application Publication
(10) Pub. No.: US 2007/0048808 A1
(43) Pub. Date: Mar. 1, 2007

(54) Hepatocellular carcinoma screening

(76) Inventor: Mark A. Feitelson, North Wales, PA (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(21) Appl. No.: 11/497,155

(22) Filed: Jul. 31, 2006

Related U.S. Application Data
(63) Continuation-in-part of application No. 09/523,389, filed on Mar. 10, 2000, now abandoned.
(60) Provisional application No. 60/124,284, filed on Mar. 12, 1999.

(51) Int. Cl. G01N 33/574

(52) U.S. Cl. 435/7.23

(57) ABSTRACT

A method for identifying individuals at risk for developing hepatocellular carcinoma is described. The method detects differential patterns of gene expression that are caused by the presence of hepatitis B virus x antigen.

### CROSS REFERENCE TO RELATED APPLICATIONS

The present application is a continuation-in-part of copending U.S. patent application Ser. No. 09/523,389, filed Mar. 10, 2000, which application is entitled to priority under 35 U.S.C. § 119(e) to U.S. Provisional Application No. 60/124,284, filed Mar. 12, 1999, each of which application is incorporated by reference herein in its entirety.

### REFERENCE TO GOVERNMENT GRANT

This invention was made in the course of research sponsored by the National Institutes of Health grants CA48656 and CA66971. The U.S. Government has certain rights in the invention.

### FIELD OF THE INVENTION

The invention is directed to diagnostic screening, particularly diagnostic screening for identifying individuals afflicted with hepatocellular carcinoma or at risk of developing hepatocellular carcinoma.

### BACKGROUND OF THE INVENTION

Primary hepatocellular carcinoma (HCC) is one of the most common tumors seen in certain areas of the world. Between 250,000 and 1 million new cases are reported each year. Less than 3% of patients diagnosed with HCC survive ten years. In Asia and sub-Saharan Africa it has an annual incidence rate of 500 cases per 100,000 population. In the United States and Europe, HCC accounts for 1 to 2 percent of tumors seen at autopsy (Podolsky, D. K. and K. J. Isselbacher. 1994, Harrison's Principles of Internal Medicine, pp. 1496-1497). There are risk factors for HCC, however, that can lead to a large increase in the likelihood that tumors will develop. For example, HCC is usually associated with a cirrhotic liver, making alcoholics more likely to develop these tumors.

HCC is one of the ten most frequent cancers worldwide, with Although an efficacious vaccine is used in this country, the majority of cases occurs in the third world, where the vaccine is not available and the diagnostic and treatment options are limited. HCC therefore is a severe threat to public health.

The increased incidence of HFCC in Asian and African populations and elsewhere has been attributed to the high incidence of chronic infection with hepatitis B virus (HBV) and hepatitis C virus (HCV). These chronic infections can lead to hepatitis and cirrhosis which are the most common risk factors for HCC. The link between HBV infection and HCC is well established At least 300 million carriers of HBV are at an increased risk for developing other liver problems, including HCC. Studies in Asia have shown that the incidence of this form of cancer over time is increased 100-fold in individuals with evidence of HBV infection as compared to non-infected controls (Podolsky et al. 1994, Harrison's Principles of Internal Medicine, pp. 1496-1497). More recent work in Europe and Japan has shown that HCV is also linked to an increased risk of HCC. In fact, any agent or factor that contributes to chronic, low-grade liver cell damage would make liver cell DNA more susceptible to damage and genetic alterations which can lead to carcinogenesis. The mechanisms and steps responsible for the development of HCC, however, have not been fully elucidated.

The finding that HBV makes a genetic contribution to the development of HCC (Seeger et al. 1991. J. Virol. 65:1673-1679) suggests that one or more virus encoded proteins may play a role in hepatocarcinogenesis. Other data suggests that hepatitis B x antigen (HBxAg) contributes to the pathogenesis of HCC. HBxAg transforms a mouse hepatocyte cell line both in vitro and in vivo (Hohne, M. et al. 1990. EMBO J. 9:1137-1145; Seifer et al. 1991. J Hepatol. 13:S61-S65). HBxAg binds to and functionally inactivates the tumor suppressor p53 (Feitelson et al. 1993. Oncogene 8:1109-1117; Wang et al. 1994. Proc. Natl. Acad. Sci. USA 91:2230-2234; Truant et al. J. Virol. 69:1851-1859; Takeda et al. 1995. J Cancel Res. Clin. Oncol. 121:593-601). HBxAg/p53 staining and complex formation has also been shown to correlate with the development of liver tumors in a X transgenic mouse model with sustained high levels of HBxAg expression (Kim et al. 1991. Nature 351:317-320; Koike et al. 1994. Hepatology 19:810-819; Ueda et al. 1995. Nature Genetics 9:41-47).

It has previously been shown that HBxAg is a trans-activating protein (Twu et al. 1987. J. Virol. 61:3448-3453; Rossner. 1992. J Med. Virol. 36:101-117; Henkler et al. 1996. J. Viral Hepatitis 3:109-121). Even though virus DNA fragments integrated into HCC cells often contain the X region (Matsubara, K. and T. Tokino. 1990. Mol. Biol. Med. 7:243-260; Unsal et al. 1994. Proc. Natl. Acad. Sci. USA 91:822-826) and HBxAg made from these integrated sequences has transactivating-activity, it is not clear that this action is responsible for transformation (Luber et al. 1996. Oncogene 12:1597-1608). A variety of studies have described differences in gene expression which distinguish tumor (HCC) form nontumor (liver) cells (Begum et al. 1995. Hepatology 22:1447-1455; Darabi et al. 1995. Cancer Lett. 95:153-159; Inui et al. 1994. Gastroenterology 107:1799-1804; Kim et al. 1996. Cancer Res. 56:3831-3836; Ohmachi et al. 1994. J. Hepatol. 21:1012-1016; DuBois 1994. Hepatology 19:788-799; Ueki et al. 1997. Hepatology 25:862-866; Yamashita et al. 1996. Hepatology 24:1437-1440; Zhou et al. 1994. Arch. Virol. 134:369-378). However, no indication has been given whether any of these genes are turned on or off by HBxAg.

The treatment of HCC is much more successful when the cancer is caught early. Survival rates are greatly increased if treatment is initiated when tumors are less than 3 centimeters. Therefore, early detection is very important in order to increase patients' chances for survival. Detection of HCC may escape clinical recognition because of the presence of other active disease processes, such as hepatitis or cirrhosis. One screening tool has been alpha fetoprotein levels, where levels greater than 500 μg/L are found in 70-80% of patients with HCC (Podolsky, D. K. and K. J. Isselbacher. 1994. Harrison's Principles of Internal Medicine, pp. 1496-1497). The most common diagnostic tools are imaging with ultrasound, which can only detect the presence of visible tumors, and liver biopsy. Neither of these diagnostic tools is able to screen individuals for the risk of disease before tumors develop. In biopsy, it can be difficult to distinguish large cirrhotic nodules from well-differentiated HCC or low-grade dysplastic nodules from HCC. Moreover, ultrasound and liver biopsy are expensive and not widely available in the third world, where the majority of cases occurs. A new, inexpensive, and specific indicator of HCC is crucial to improving the diagnosis, treatment and prognosis of HBV carriers that develop HCC. Clearly, there is a need for better methods of early diagnosis, as well as risk screening. Criteria for judging the usefulness of HCC screening methods were recently reviewed by Collier and Sherman, 1988. Hepatology 27:273-278.

### SUMMARY OF THE INVENTION

A method for identifying individuals at risk for hepatocellular carcinoma (HCC) is provided. In an embodiment, a method includes testing a blood sample for one or more marker proteins in the sample, or for one or more marker antibodies which bind to the marker proteins, which marker protein is the product of a cellular gene which is differentially expressed in HBxAg[+] cells as compared with HBxAg[−] cells. The presence of one or more marker proteins or marker antibodies in the sample is indicative of a risk for developing HCC, wherein the marker proteins encoded by nucleic acids selected from the group consisting of SEQ ID NO:1, SEQ ID NO:2, SEQ ID NO:3, SEQ ID NO:4, SEQ ID NO:5, and SEQ ID NO:9.

In an embodiment of the invention, a blood sample is tested for one or more marker proteins in the sample by immunoassay by contacting the sample with one or more antibodies which bind the marker proteins. The immunoassay may be a radioimmunoassay, an immunofluorescence assay, a chemiluminescence assay or an enzyme-linked immunosorbent assay, among others.

A method is also provided for identifying individuals at risk for HCC, the method comprising testing a blood sample for one or more marker proteins in the sample, or for one or more marker antibodies which bind to the marker proteins, which marker protein is the product of a cellular gene which is differentially expressed in HBxAg[+] cells as compared with HBxAg[−] cells. The presence of the marker proteins or marker antibodies in the sample is indicative of a risk for developing hepatocellular carcinoma, wherein the marker proteins are encoded by nucleic acids selected from the group consisting of SEQ ID NO:1, SEQ ID NO:2, SEQ ID NO:3, SEQ ID NO:4, SEQ ID NO:5, and SEQ ID NO:9, and the antibodies bind to an antigen of one or more of the marker proteins. In one embodiment, the blood sample is contacted with an immunoreagent comprising one or more peptides selected from the group consisting of SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, and SEQ ID NO:26.

A method is also provided for identifying individuals at risk for HCC, comprising testing a blood sample for one or more marker antibodies which bind to the marker proteins, which marker protein is the product of a cellular gene which is differentially expressed in HBxAg[+P] cells as compared with HBxAg[−] cells. The presence of the marker antibodies in the sample is indicative of a risk for developing hepatocellular carcinoma, wherein the blood sample is contacted with an immunoreagent comprising one or more peptides selected from the group consisting of SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, and SEQ ID NO:26.

### BRIEF DESCRIPTION OF THE DRAWINGS

FIG. 1 is a graph of the percentage of patients testing positive for five antibodies: 1, anti-C2 (light gray bars); 2, anti-L4 (white bars); 3, anti-L7 (black bars); 4, anti-L11 (dark gray bars); 5, anti-L12 (patterned bars). The patients are in three groups: normal blood donors; HBV patients who did not have HCC; and HBV patients who were afflicted with HCC.

### DETAILED DESCRIPTION OF THE INVENTION

A. Abbreviations

“bp” base pair

“CAT” chloramphenicol acetyltransferase

“ELISA” enzyme-linked immunosorbent assay

“HBxAg” hepatitis B x antigen

“HBsAg” hepatitis B surface antigen

“HBV” hepatitis 13 virus

“HCC” hepatocellular carcinoma

“PCR” polymerase chain reaction

“RT” reverse transcriptase

“SSC” standard saline citrate solution (0.15M saline containing 0.015M sodium citrate, pH 7)

B. Definitions

“Expression” means, with respect to a gene, the realization of genetic information encoded in the gene to produce a functional RNA or protein. The term is thus used in its broadest sense, unless indicated to the contrary, to include either transcription or translation.

“Hybridization” means the Watson-Crick base-pairing of essentially complementary nucleotide sequences (polymers of nucleic acids) to form a double-stranded molecule.

By “immunoreagent” is meant herein and antigen or antibody which is used to detect the presence of a complementary immunoreagent in a biological sample. The complementary immunoreagent binds the immunoreagent through an antigen-antibody binding reaction to form an immunocomplex.

“Marker gene” means a gene which is differentially expressed in HBxAg[+] cells as compared to HBxAg[−] cells.

By “marker protein” is meant the protein expression product of a marker gene.

By “marker antibody” is meant an antibody generated by the immune system of the host in response to marker gene expression.

### DETAILED DESCRIPTION OF THE INVENTION

According to the invention, a screening procedure is provided for identifying individuals at risk for developing HCC. Marker genes which are differentially expressed in HBxAg[+] cells as compared to HBxAg[−] cells encode marker proteins which may appear in the blood of individuals at risk for developing HCC. Antibodies generated by the host against these inappropriately expressed proteins may also be detected in the blood of patients at risk of developing HCC. The marker genes and their expression products are more fully characterized below.

It has been found that normal individuals do not possess antibodies to any of the marker proteins in their blood. HBV-positive patients whose HBV infection is non-chronic likewise lack the marker antibodies in their blood. Typically, non-chronic HBV-infected individuals are at low risk of developing HCC. Hence, the absence of marker antibodies correlates with a low risk of developing HCC.

It is known that chronic HBV-infected patients are at substantial risk of developing HCC. It has been found chronic HBV-infected individuals typically contain at least one of the marker antibodies in their blood. Hence, the presence of one or more marker antibodies (or marker proteins) correlates with substantial risk of developing HCC. In the studies that follow, many pre-HCC patients displaying marker proteins in their blood were ultimately diagnosed as having HCC. In some cases, a high antibody level correlated with disease stage, that is, the highest levels of marker antibody against some marker proteins were found in active HCC patients as opposed to chronic HBV-infected individuals who did not yet have the disease.

The method of the present invention is useful as a screen for identifying individuals at risk of developing HCC. Those individuals testing positive for marker protein or marker antibody may then be closely monitored for the appearance of tumors by ultrasound or other means. Early detection of tumors permits early treatment and improved prognosis. Survival rates are greatly increased if treatment is initiated when tumors are less than 3 centimeters.

The screening procedure to detect either marker protein or marker antibody most conveniently takes the form of an immunoassay. According to one embodiment, the screening procedure detects host antibodies in the blood which are produced against the inappropriately expressed marker proteins. In this form of antigen-antibody assay, the immunoreagent comprises an antigen, and the complementary immunoreagent to be detected in the sample comprises an antibody. The immunoreagent may be a substantially complete marker protein. More preferably, the immunoreagent takes the form of an antigenic fragment of a marker protein, or a synthetic peptide modeled after such a fragment. Thus, the amino acid sequence of the marker protein may be used to design antigen peptides for use as immunoreagents in identifying antibodies against marker proteins in the blood.

According to another embodiment, the assay is designed to detect the presence of marker protein in the blood. In the case of an immunoassay, the immunoreagent comprises an antibody to the marker protein of interest.

The antigen or antibody immunoreagents are conveniently labeled. A detectable label may be directly attached to the immunoreagent. More conveniently, the detectable label is attached to a second immunoreagent antibody which binds either the first immunoreagent or the complementary immunoreagent in the immunocomplex.

The marker genes were identified by manipulation of HepG2X cells. HepG2 is a differentiated cell line derived from a human hepatoblastoma. The cell line HepG2X was generated by infection of HepG2 cells by replication defective recombinant retroviruses encoding the fall length HBxAg polypeptide. HepG2CAT cells were generated in the same manner by substituting the bacterial CAT gene for the HBV X gene in the transfection vector. The HepG2X cells express the HBV X antigen (HBxAg[+]), while HepG2CAT cells do not (HBxAg[−]).

Genes whose expression are either turned on or of in the presence of the hepatitis B x antigen (HBxAg) in HepG2 cells ale identified by PCR select cDNA subtraction. Briefly, the method consists of isolating whole cell RNA from HBxAg [+] and [−] HepG2 cells. Methods and kits for performing PCR select cDNA subtraction are well-known and commercially available, e.g., from Clontech, Palo Alto, Calif. The RNA from HepG2X cells is subtracted from those in HepG2 cells, providing RNAs expressed in HepG2X cells but not in HepG2 cells. The RNAs are then reverse transcribed into DNA and then PCR amplified using random primers. In order to obtain RNAs expressed in HepG2, but not HepG2X cells, the opposite subtraction is carried out. These RT/PCR fragments are then cloned and either partially or fully sequenced.

Accordingly, equivalent amounts of poly(A)+ RNA were isolated from confluent cultures of HepG2X and HepG2CAT cells and subjected to PCR select cDNA subtraction. DNA strands were individually sequenced from every clone, and the results for each compared to entries in GenBank and other related databases (Table 1, below). The PCR select cDNA subtraction generated gene fragments from different cellular genes that were detected in HepG2X cells but not in HepG2CAT cells. These fragments are referred to herein as L7, L8, L12, L16 and L19. Some of these fragments had at least 95.7% homology with fragments of known products from GenBank: L7 shares homology with H49417 (SEQ ID NO:1), L8 shares homology with U61232 (SEQ ID NO:2), L12 shares homology with S15A (P48149 (Swissprot), corresponding to GenBank nucleotide reference number Z21673) (SEQ ID NO:3), L16 shares homology with d50922 (SEQ ID NO:4) and L19 shares homology with AA026758 (SEQ ID NO:5) (see Table 1). Interestingly, three of the five sequences (L7, L12, and L19) had homology with factors upregulated in fetal tissues, suggesting that they may have some growth regulatory functions. In addition, a fragment, C2, which shares homology with GenBank accession number L26247 (SEQ ID NO:9), was apparently present in HepG2CAT cells but absent in HepG2X cells. All of the aforementioned genes were differentially expressed in HepG2X compared to HepG2CAT cells. In the case of the transcripts hybridizing to the “L” fragments, the clones represent fragments of genes whose expression is activated in HBx/Ag[+] cells compared to HBxAg[−] cells. In the case of transcripts hybridizing to the “C” fragments, the clones represent genes whose expression is suppressed in HBxAg[+] cells compared to HBxAg[−] cells. The fragment size given in Table 1 is considered approximate, as size was estimated visually from gels.

TABLE 1
Differentially expressed genes in HBxAg[+] and [−] Hep G2 cells

| | insert | GenBank Search | |
|---|---|---|---|
| | size | Match (and | % homology |
| clone | (˜bp) | Accession #) | in overlap |
| HBx[+]^{a} | | | |
| Clones | | | |
| L7 | (690) | human fetal liver | 95.7% in 440 bp |
| | | cDNA clone (H49417) | |
| L8^{b} | (220) | human tubulin- | 100% in 45 bp |
| | | folding cofactor E | |
| | | cDNA (U61232) | |
| L12 | (320) | human 40S ribosomal | — |
| | | protein S15A (P48149) | |
| L16^{b} | (180) | human myeloblast | 99% in 65 bp |
| | | KIAA0132 gene | |
| | | (d50922) | |
| L19 | (250) | human fetal heart | 99% in 152 bp |
| | | cDNA (AA026758) | |
| L4^{b} | (1700) | none | — |
| L11 | (580) | none | — |
| L15 | (1580) | none | — |
| HBx[−]^{a} | | | |
| Clones | | | |
| C2^{b} | (620) | human sui1 (L26247) | — |
| C1 | (670) | none | — |

^{a}The clones represent fragments of genes whose expression is activated (L7, L8, L12, L16, L19, L4, L11 and L5) or suppressed (C2, C1) in HBxAg[+] compared to HBxAg[−] cells.
^{b}Probes whose sequences share considerable homology with sequences independently found in tumor compared to nontumor cells.

The cDNA fragments obtained front subtraction hybridization (Table 1) were used as probes for ISH of HepG2X and HepG2CAT cells, to verify that the probes obtained from PCR select cDNA subtraction actually represented differentially expressed genes in HepG2 compared to HepG2X cells. In all cases, the L probes hybridized to HepG2X cells. Little or no signal was observed in HepG2CAT cells. In contrast, the C probes demonstrated strong hybridization in HepG2CAT cells, but little or no signal in HepG2X cells. Thus, in all cases, the probes obtained from PCR select cDNA subtraction actually reflected differences in gene expression between HepG2X and HepG2CAT cells. The cDNA fragments were either partially or completely sequenced.

In order to further study the structure and function of the protein encoded by the C2 mRNA, the full length cDNA containing the C2 sequence was obtained (from HepG2CAT cells) by 5′ and 3′ rapid amplification of cDNA ends (RACE) PCR using the Marathon™ cDNA Amplification Kit (Clontech, Palo Alto, Calif.). Briefly, one 3′ and one 5′ gene specific primers were synthesized. PCR was performed using these primers together with an adaptor primer to obtain the 3′ or 5′ cDNA specific products in separate amplification reactions. The products were cloned into pT7Blue T (Novagen, Inc., Madison, Wis.) and sequenced. The appropriate 3′ and 5′ gene specific fragments were then digested with suitable restriction enzymes and cloned into pcDNA3 (Invitrogen, San Diego, Calif.) at the chosen site(s), and the integrity of the full length clone verified by DNA sequencing. This resulted in a lull length clone exactly 1.35 kb in length, which encoded a small protein of 113 amino acids near its 5′ end that has 100% homology with the human translation initiation factor, hu-sui1 (accession no. L26247, SEQ ID NO:9). The C2 probe spans bases 903-1350 of full length hu-sui1 cDNA.

Other than its regulatory role in translation initiation, the human Siu1 protein does not appear to have any recognizable motifs which would suggest additional functions. These results indicate that the introduction of HBxAg results in the altered expression of a protein whose function is associated with the regulation of translation.

Additional full length cDNAs from differentially expressed genes containing fragments L7 and L12 were obtained in a similar manner to fragment C2. The cDNA containing fragment L12 encoded a protein of 130 amino acids having a 100% homology with the human 40S ribosomal protein S15A (Accession nos. P39027, P39031). Sequences of the full length cDNAs and corresponding gene names are set forth in Table 2:

TABLE 2
Nuclcotidc Sequence Homology of Full-length
Differentially Expressed cDNAs

| cDNA | | GenBank Accession Number |
|---|---|---|
| Fragment | Gene Name | of Homologous Sequence |
| L7 | human fetal liver cDNA | H49417 |
| L12 | human 40S ribosomal | P48149 |
| | protein S15A | |
| C2 | hu-sui1 | L26247 |

cDNA fragments for two separate unknown proteins were also identified. These cDNA fragments were referred to as L4 and L11, repsectively.

The remaining full length cDNAs corresponding to cDNA fragments L8, L16, and L19 may be obtained in the same manner, or the cDNA sequence may be obtained through GenBank from the accession numbers provided in Table 1.

For purposes of an immunoassay for detecting a blood marker antibody, the antigen immunoreagent may comprise full length marker protein. The marker protein may be obtained in the appropriate quantities through recombinant DNA techniques known to those skilled in the art based upon the foregoing identification of the corresponding marker genes. Preferably, the immunoreagent for the detection of marker antibody in the blood of test subjects comprises antigenic peptides derived from the structure of the full length marker protein. Hydrophilic regions, which face the environment surrounding the protein, are most likely to contain antigenic sites. Such regions can be identified in the amino acid sequence of the marker protein using standard computer programs.

For purposes of an immunoassay for detecting a blood marker antigen, the antibody immunoreagent may comprise monoclonal or polyclonal antisera raised against the marker antigen. The antibody may comprise an intact antibody, or fragments thereof capable of specifically binding marker protein. Such fragments include, but are not limited to, Fab and F(ab′)₂ fragments. As used herein, the term “antibody” includes both polyclonal and monoclonal antibodies. The term “antibody” means not only intact antibody molecules, but also includes fragments thereof which retain antigen binding ability.

Appropriate polyclonal antisera may be prepared by immunizing appropriate host animals with marker protein and collecting and purifying the antisera according to conventional techniques known to those skilled in the art. Monoclonal antibody may be prepared by following the classical technique of Kohler and Milstein, Nature 254:493-497 (1975), as further elaborated in later works such as Monoclonal Antibodies, Hybridomas: A New Dimension in Biological Analysis, R. H. Kennet et al., eds., Plenum Press, New York and London (1980).

Substantially pure marker protein for use as an immunogen for raising polyclonal or monoclonal antibodies may be conveniently prepared by recombinant DNA methods.

As an alternative to immunization with the complete marker protein, antibody against marker proteins can be raised by immunizing appropriate hosts with immunogenic fragments of the whole protein, particularly peptides corresponding to probable antigenic determinants. Hydrophilic regions, which face the environment surrounding the protein, are most likely to contain antigenic sites. As indicated above, these regions can be identified using standard computer programs.

The test sample may comprise whole blood. More conveniently, the test sample comprises serum. As used herein, a “blood sample” means not only whole blood, but a sample consisting of a blood traction, such as serum. Serum samples may be obtained from whole blood draws by well-known serum preparation techniques.

According to a preferred embodiment, the invention comprises a solid phase immunoassay. An immunoreagent (marker antibody or marker antigen) is adhered to a solid support. The immobilized immunoreagent is contacted with a liquid comprising or containing the biological test sample. The sample and immobilized immunoreagent are incubated for a time sufficient to permit formation of an immunocomplex of the immunoreagent and its complementary immunoreagent. The time for an ELISA should not be less than about 1.5 hours for each incubation step of antibody with antigen. Longer times may be employed.

After the immobilized immunocomplex is formed, either with or without intervening wash steps, it is contacted with a second immunoreagent which bears a detectable label. The second immunoreagent may comprise either an antigen or an antibody, but typically comprises an antibody. The second immunoreagent is labeled directly with the detectable label, or is labeled indirectly through coupling to another molecule which bears the detectable label. The label may advantageously comprise, for example, a radionuclide in the case of a radioimmunoassay; a fluorescent moiety in the case of an immunofluorescence assay; a chemiluminescent moiety in the case of a chemiluminescence assay; and an enzyme which cleaves a chromogenic or fluorogenic substrate, in the case of an enzyme-linked immunosorbent assay (ELISA). Preferably, the assay takes the form of an ELISA.

Following one or more washing steps to remove any unbound material in an enzyme immunoassay, an indicator substance, for example, a chromogenic substrate, is added which reacts with the enzyme to produce a color change. The color change can be observed visually or more preferably by an instrument to indicate the presence or absence of an antibody or antigen in the sample. For quantification, an instrument is required. For solid phase fluorescence immunoassays, fluorescent labeled moieties can be monitored using excitation at an appropriate wavelength, while chemiluminescent labeled antigens or antibodies can be followed after reaction by chemically activating the chemiluminescent labels to generate light which can be detected by photometric means.

The underlying principle of an immunoassay is that the concentration of the antigen-antibody complex is proportional to the concentration of free antigen and free antibody in the assay medium. Thus, a calibration curve for the determination of an antigen (or antibody) can be constructed by measuring the amount of antigen-antibody complex formed upon addition of varying and known amounts of antigen (or antibody) to a solution containing a fixed and known amount of antibody (or antigen).

The screening procedure described herein may take the form of detecting the presence of just one marker antibody, e.g., marker antibody which binds to protein hu-Sui-1. Hence, the immunoreagent for detecting the target marker antibody will comprise hu-Sui-1 protein or antigenic fragment thereof. Alternatively, a mixture of immunoreagent species targeting different marker antibodies may be utilized, e.g., a mixture comprising hu-Sui-1 and L7 proteins, or antigenic fragments thereof. By testing for the presence of multiple marker antibodies in this manner, it is expected that the sensitivity of the assay will be increased. It should be appreciated that even where only one marker antibody is targeted for detection, the test reagent may comprise a plurality of antigenic fragments of the marker protein. For example, the immunoreagent for detecting anti-hu-Sui-1 antibody may comprise a mixture of peptides C2.1 (SEQ ID NO:16) and C2.2 (SEQ ID NO:17), corresponding respectively to hu-Sui-1 amino acids 52-69 and 75-94.

Similarly, where the screening procedure takes the form of detection of marker protein antigens, the presence of just one marker protein, e.g. hu-Sui-1, may be determined. Hence, the immunoreagent for detecting the target marker protein will comprise anti-hu-Sui-1 antibody. Alternatively, a mixture of immunoreagent species targeting different marker proteins may be utilized, e.g., a mixture comprising anti-hu-Sui-1 and anti-L7 antibodies. By testing for the presence of multiple marker proteins in this manner, it is expected that the sensitivity of the assay will be increased. It should be appreciated that even where only one marker protein is targeted for detection, the test reagent may comprise a single polyclonal or monoclonal antisera, or pooled antisera from one or more hosts immunized with the same antigen.

Preferably, the screening method is constructed so as to detect multiple marker antibodies (or multiple marker antigens) in the blood of the test subject. Thus, a mixture of immunoreagents is utilized. Preferably, the mixture contains at least one hu-Sui1 reagent, that is, an hu-Sui1 antigenic peptide in the case of a screen for marker antibodies, or an hu-Sui1 antibody in the case of a screen for detection of marker proteins. Preferred marker proteins for screening include C2 (which shares homology with hu-Sui1 (SEQ ID NO:9)) and L7 (which shares homology with SEQ ID NO:1). Likewise, preferred antibodies for screening include anti-C2 and anti-L7.

The following non-limiting examples are provided to illustrate the claimed invention.

### EXAMPLE 1

### Preparation of HBxAg[+] and HBxAg[−] Cell Lines

A. Cell Lines and Culture Conditions

HepG2 cells, a differentiated cell line derived from a human hepatoblastoma (Aden, D. P. et al. 1979. Nature 282:615-617; Knowles, B. B. et al. 1980. Science 209:497-499), were cultured on type-1 rat tail collagen (Becton Dickinson, Franklin Lakes, N.J.) coated tissue culture dishes or plates. Cells were grown in Earle's MEM supplemented with 10% heat inactivated fetal calf serum (FCS), 100 μM MEM non-essential amino acids, 1 mM sodium pyruvate, as well as standard concentrations of penicillin plus streptomycin. The retrovirus packaging cell line PA317 (Danos, O. 1991. Methods in Molecular Biology, Practical Molecular Virology: Viral Sectors for Gene Expression 8:17-27) was also grown on plastic dishes in the same medium.

B. Plasmid Construction

The retroviral vector plasmid, pSLXCMVneo, was used to clone the HBV X gene (Valenzeula, P. et al. 1980. Animal Virus Genetics, Academic Press: New York, pp. 57-70) or the bacterial chloramphenicol acetyltransferase (CAT) gene sequences for these studies, as described (Duan, L. X. et al. 1995. Human Gene Ther. 6:561-573). Briefly, pSLXCMV-CAT was constructed by inserting a 726 bp HindIII-BamHI fragment containing the CAT gene into the HpaI-BgIII site of the pSLXCMV polylinker. PSLXCMV-FLAG-HBx was constructed by inserting a 920 bp MluI-BglII fragment of FLAG-HBx DNA into the MluI-BglII site of the pSLX-CMV polylinker. Recombinants were used to transform HB101. Minipreps were prepared and the DNA used for sequence analysis.

C. Preparation of Recombinant Retroviruses and Infection of HepG2 Cells

Approximately 1×10⁶ PA317 cells/100 mm dish were transfected using standard calcium phosphate precipitation using 15 μg of pSLXCMV-FLAG-HBx or 15 μg of pSLXCMV-CAT. At 24, 48, and 72 hours after transfection, the medium was removed and processed through a 0.45 μm filter to remove PA317 cells, and then used immediately for infection of HepG2 cells. Five ml of recombinant retrovirus-enriched supernatant (5×10⁵ CFU/ml, as assayed on NIH-31T3 cells) was used to infect 1×10⁶ target HepG2 cells/100 mm dish in the presence of polybrene (8 μg/ml) for 24 hours. Fresh virus supernatant was added after 24 and again after 48 hours so that the cells were exposed to virus for a total of 72 hours. All of these infections were carried out in log phase cultures. Cells were then passaged at 1:2 and selected by incubation in G418 (800 μg/ml; GIBCO/BRL, Grand Island, N.Y.) for 14 days in order to maximize the fraction of cells producing HBxAg or CAT. G418 colonies were then expanded in normal growth medium and used for analysis. The fourteen day selection in G418 had the effect of eliminating most of the uninfected cells.

D. Detection of CAT Activity and HBxAg Polypeptide in Transfectants

The transfectants (HepG2-CAT and HepG2X) were evaluated as follows.

CAT assays were performed as described by Wang et al. (1994. Proc. Natl. Acad. Sci. USA 91:2230-2234). Briefly, 1×10⁷ HepG2-CAT cells in a 100 mm dish were lysed by addition of 0.9 ml of 1× report lysis buffer (Promega) for 15 minutes and harvested by scraping. Cells were pelleted and 180 μl of cell lysate was used for a standard CAT assay. After incubation with ¹⁴C-chloramphenicol, acetylated forms were separated by thin-layer chromatography. Alternatively, lysates prepared from 5×10⁶ HepG2X cells were assayed for the 17 kDa HBxAg by western blotting using a mixture well characterized rabbit anti-x peptide antibodies (Feitelson, M. A. and M. M. Clayton. 1990. Virology 177:367-371; Feitelson, M. A. et al. 1990. Gastroenterology 98:1071-1078). Horseradish peroxidase conjugated goat anti-rabbit Ig (Accurate, Westbury, N.Y.) and ECL substrate (Amersham, Arlington Heights, Ill.) were used for detection.

CAT activity was present in HepG2CAT, but not in HepG2X cells. HBxAg was present in lysates from HepG2X, but not from HepG2CAT cells. Together, these findings show that both of the recombinant retroviruses are expressing the expected products in HepG2 cells.

### EXAMPLE 2

### Identification of Differentially Expressed Genes Distinguishing HepG2X from HepG2CAT

The differences in gene expression which distinguish HepG2X from HepG2CAT cells were determined by using a commercially available subtraction hybridization approach (the PCR-select cDNA subtraction kit from Clontech, Palo Alto, Calif.). Briefly, whole cell RNA was extracted separately from 1×10⁷ HepG2X and an equal number of HepG2CAT cells, and the quality of the extraction was determined by assaying for 18S and 28S rRNAs by agarose gel electrophoresis and ethidium bromide staining. PCR-select cDNA subtraction is reverse transcriptase (RT)/PCR based, and enriches for poly A⁺ RNA (isolated using the Qiagen RNeasy total RNA kit; QIAGEN, Inc., Chatsworth, Calif.) from tissue culture cells or tissues. The procedure involved ligating adaptors to some of the PCR products and conducting two rounds of subtractive hybridization against the PCR products from the cells in which the comparison were being made. The resulting products were then PCR amplified using primers which matched the sequence of the adaptors (in the CLONTECH Advantage cDNA PCR kit). The unique fragments were then eluted from the gels (using the QIAGEN gel extraction kit) and cloned into pT7Blue (Novagen, Madison, Wis.). Positive clones were selected by blue-white phenotype. Recombinant DNAs were isolated from minipreps of individual clones, digested by Rsa I to check insert size, and then both strands individually analyzed by sequence analysis. The sequences obtained were then compared to those in GenBank using the FASTA command in the GCG software package for homology to known genes. The results are set forth in Table 1, above.

### EXAMPLE 3

### Preparation of Peptide and Antibody Immunoreagents for HCC Screening

Peptide immunoreagents for detection of marker antibodies, and antibody immunoreagents for detection of marker proteins, were prepared as follows.

A. Peptides

Synthetic peptides that represent probable antigenic determinants on each of the differentially expressed proteins corresponding to gene fragments L7, which shares homology with H49417 (SEQ ID NO:1), 112, which shares homology with S15A (P48149) (SEQ ID NO:3), and C2, which shares homology with GenBank accession number L26247 (SEQ ID NO:9), as well as gene fragments L4 and L11, which share homology with two separate unknown proteins, as described elsewhere herein, were prepared by solid phase peptide synthesis and analyzed by HPLC and amino acid composition prior to use. The peptides are identified in Table 4, below. The peptides were coupled by virtue of their free cysteine sulfhydryl (either in the peptide sequence or added to the carboxy or amino terminus where the native sequence did not contain a cysteine) to keyhole limpet hemocyanin (KLH; Sigma) using the coupling agent m-maleimidobenzyol-N-hydroxysuccinimide ester (MBS; Pierce) as described by Liu et al., Biochemistry 18:690-697 (1979).

TABLE 4

Peptide fragments of differentially
expressed proteins

| | | | Peptide |
|---|---|---|---|
| Gene | Pep- | | Position |
| Frag. | tide | Sequence | in Protein |
| C2 | C2.1 | DDYDKKKLVKAFKKKFAC | 52-69 |
| | | (SEQ ID NO:16) | |
| | C2.2 | EHPEYGEVIQLQGDQRKNIC | 75-94 |
| | | (SEQ ID NO:17) | |
| L4 | L4A | CQKAKDRMERITRKIKDSDAYRRDE | 460-484 |
| | | (SEQ ID NO:18) | |
| | L4B | PRPRDKRQLLDPPGDLSRC | 821-838 |
| | | (SEQ ID NO:19) | |
| L7 | L7A | CGVWNQTEPEPAATS | 12-25 |
| | | (SEQ ID NO:20) | |
| | L7B | HHHGRGYLRMSPLFKC | 56-70 |
| | | (SEQ ID NO:21) | |
| L11 | 1L11 | PCPELACPREEWRLGP | 2-17 |
| | | (SEQ ID NO:22) | |
| | 3L11 | DPSRSPHSTSSFPRGSSATSCDSR | 316-339 |
| | | (SEQ ID NG:23) | |
| | 4L11 | HPPDGSFSTFHDGPQPLEDPC | 359-378 |
| | | (SEQ ID NO:24) | |
| L12 | L12.1 | KSINNAEKRGKRC | 12-23 |
| | | (SEQ ID NO:25) | |
| | L12.2 | DHEERRRKHTGGKC | 112-124 |
| | | (SEQ ID NO:26) | |

B. Antibody Production

For antibody production, 5- to 10-week old female New Zealand White rabbits (2 animals/peptide; Hazelton) were bled and then injected with peptide conjugate as described (Bittle et al., Nature 298:30-33, 1982). Dilutions of immune sera were assayed in parallel with preimmune sera in solid-phase assays in wells (Immunolon 2 Removawell Strips, Dynatech Labs) coated with the appropriate (unconjugated) synthetic peptide. See Feitelson et al., Gastroenterology 98: 1071-1078, 1990 or Feitelson et al., J Med Virol 24:121-136, 1988 for additional details of the solid phase assay design.

### EXAMPLE 4

### Antibody Serum Assay for HCC Risk Factor

A. Serum Samples

Serum samples were taken from four populations. The first was a population of HBV carriers who were pre-HCC patients. This group included serial serum samples, taken repeatedly over the course of the illness, and cross-sectional samples, taken just once during the illness. A second population included Greek chronic HBV carriers, who either had HCC or did not. This population also included serial and cross-sectional samples. A third population was a cross-sectional population of HBV-negative Icelandic blood donors. The fourth population comprised a serial serum population of HBV-positive, nonchronic dialysis patients. All populations were analyzed for the presence of serum antibodies against certain marker proteins, as follows.

B. Serum Sample ELISA

Serum samples were screened for the presence of marker antibodies by (ELISA). Ninety-six well plates were coated with 50 μl of synthetic peptides from Table 4, above at a concentration of 1 μg/well, using phosphate buffered saline (PBS, pH 7.41)/10% fetal bovine serum (FBS) as the diluent. The plates were then incubated for 24 hours at 4° C. Each well contained one of the peptide sets from Table 4. In addition to the plates containing peptides, a control plate, containing only 5 μl of the diluent was also set up.

After the incubation period, the wells were washed seven times, using a Nunc 12-port Well Washer with PBS (pH 7.41). Fifty μl of the serum samples, diluted 1:10 with PBS/10% FBS, were pipetted into the corresponding wells on the plate containing peptide and the control plate lacking peptide. Positive controls, consisting of 1:50 rabbit antibody in PBS/10% FBS were added to the corresponding wells on both plates. Negative controls, consisting of commercially obtained normal human sera in a 1:10 dilution, were also employed. Blank wells, with 50 μl PBS/FBS, were included on each plate. All plates were again incubated for 24 hours at 4° C.

After incubation, the plates were washed seven times with PBS, as described above. Fifty μl of secondary antibody, goat anti-human-Ig conjugated with horseradish peroxidase, were added to each well at a concentration of 1:6400. The secondary antibody for the positive controls (rabbit antibody) was goat anti-rabbit-Ig at a concentration of 1:1600. All plates were incubated at 37° C. for one hour. The plates were then washed seven times with P13S and 50 μl of substrate was pipetted into each well. The substrate consisted of one 15 mg tablet of O-phenylenediamine dihydrochloride dissolved in 20 ml of buffer. The buffer was made by adding one tablet of phosphate-citrate buffer with urea hydrogen peroxide to 100 ml deionized water. Alter the substrate was added, the plates were read spectrophotometrically at 450 nm on a Dynatech MR5000 ELISA Plate Rcader at 5, 10, and 15 minutes. The samples were analyzed for the presence of marker antibody: a sample was considered positive if the optical density was greater than the mean plus two times the standard deviation of the negative control samples. If the blank control plate also read positive for a given sample, the sample was considered negative for antibody.

C. Results—Cross-sectional Populations

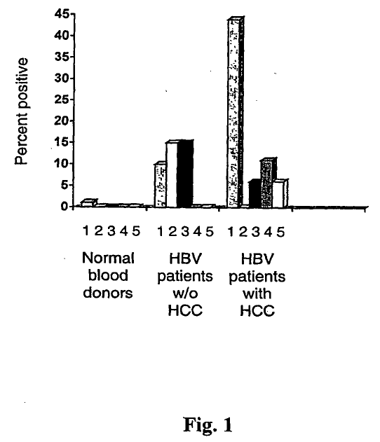

FIG. 1 represents the data from the cross-sectional populations. The y axis is the percent of serum samples that was positive for a given antibody. The x axis represents the three cross-sectional populations. The results are also summarized in Table 5:

TABLE 5
Percent of Serum Samples Positive for Antibody

| | Normal Icelandic | | Greek | | Greek | |
|---|---|---|---|---|---|---|
| | Blood Donors | | HBV+ | HCC− | HBV+ | HCC+ |
| | No. of Samples | | | | | |
| | 118 | | 20 | | 18 | |
| | % | # | % | # | % | # |
| Antibody | positive | positive | positive | positive | positive | positive |
| xC2 | 1% | 1 | 10% | 2 | 44% | 8 |
| xL4 | 0% | 0 | 15% | 3 | 0% | 0 |
| xL7 | 0% | 0 | 15% | 3 | 6% | 1 |
| xL11 | 0% | 0 | 0% | 0 | 11% | 2 |
| xL12 | 0% | 0 | 0% | 0 | 6% | 1 |

The normal (Icelandic) blood donor sera contained almost no marker antibodies; only one patient was positive for one antibody, anti-C2. The Greek population of chronic HBV carriers who had not yet developed HCC showed elevated levels of anti-C2, anti-L4 and anti-L7. They did not contain any antibodies to L11 and L12. The Greek population of HBV carriers that also had HCC showed a different pattern of antibody expression. Anti-C2 levels were much higher than the cancer-free HBV carriers. Anti-L4 antibodies were not present in HCC patients. The levels of the three remaining antibodies, anti-L7, anti-L11 and anti-L12, was near 10% in the HCC patients.

The HBV-positive, non-chronic dialysis patients showed no antibody expression (data not shown).

The results indicate a clear association between the development of HCC and elevated antibody levels. It appears from the data that as HBV infection becomes chronic, at least anti-C2 antibody appears, which becomes even further elevated when HCC develops.

D. Results—Serial Populations

The results of a longitudinal analysis of the serial Greek chronic HBV carriers are set forth in Table 6. These individuals, comprising HFCC and pre-HCC patients, show elevated levels of marker antibody expression, indicating an association between development of HCC and elevated antibody levels. The (*) in Table 6 denotes the point in time where the diagnosis of HCC (“HCC dx”) was made. In these longitudinal studies, the data suggest that in some patients who develop HCC within the observation period when serum samples were collected (labeled as “months”) have one or more antibody specificities that precede the time of diagnosis (patients 1, 3, 5, 9, 10, 11). Patient 2 developed HCC at month 15 after the beginning of observation, but did not develop antibodies. The presence of antibodies in patients 4, 6, 7, and 8 suggest that they are also at increased risk for the development of liver cancer, and should be monitored closely.

TABLE 6
Longitudinal Study Results - Chronic Carriers of HBV

Patient 1

| Anti-C2 | − | − | − | − | − | − | − | − | − | − | − | − | − | − | − | − |
|---|---|---|---|---|---|---|---|---|---|---|---|---|---|---|---|---|
| Anti-L4 | − | − | − | − | − | − | − | − | − | − | − | − | − | − | − | − |
| Anti-L7 | − | − | − | − | − | − | − | − | − | + | + | + | + | + | + | + |
| Anti-L11 | − | − | − | − | − | − | − | − | − | − | − | − | − | − | − | − |
| Anti-L12 | − | − | − | − | − | − | − | − | − | − | − | − | − | − | − | + |
| HCC dx (*) | | | | | | | | | | | * | | | | | |
| Months | 0 | 3 | 6 | 9 | 12 | 15 | 18 | 21 | 23 | 24 | 26 | 27 | 28 | 29 | 32 | 33 |
| Patient 2 | | | | | | | | | | | | | | | | |

| Anti-C2 | − | − | − | − | − | − | − | − |
|---|---|---|---|---|---|---|---|---|
| Anti-L4 | − | − | − | − | − | − | − | − |
| Anti-L7 | − | − | − | − | − | − | − | − |
| Anti-L11 | − | − | − | − | − | − | − | − |
| Anti-L12 | − | − | − | − | − | − | − | − |
| HCC dx (*) | | | | * | | | | |
| Months | 0 | 6 | 10 | 15 | 18 | 20 | 21 | 26 |
| Patient 3 | | | | | | | | |

| Anti-C2 | − | − | − | − | − | − | − | − | − | − | − | − | − |
|---|---|---|---|---|---|---|---|---|---|---|---|---|---|
| Anti-L4 | − | − | − | − | − | − | − | − | − | − | − | − | − |
| Anti-L7 | − | − | − | − | − | − | − | − | − | − | − | − | − |
| Anti-L11 | − | − | + | + | + | + | + | − | − | + | + | + | + |
| Anti-L12 | − | − | − | − | − | − | − | − | − | − | − | − | − |
| HCC dx (*) | | | | * | | | | | | | | | |
| Months | 0 | 22 | 30 | 36 | 38 | 40 | 42 | 45 | 48 | 52 | 53 | 58 | 59 |
| Patient 4 | | | | | | | | | | | | | |

| Anti-C2 | − | − | − | − | − | − | − | − | − |
|---|---|---|---|---|---|---|---|---|---|
| Anti-L4 | − | − | − | − | − | − | − | − | − |
| Anti-L7 | − | − | − | − | − | − | − | − | − |
| Anti-L11 | − | − | − | + | + | + | + | + | + |
| Anti-L12 | − | − | − | − | − | − | − | − | − |
| HCC dx (*) | | | | | | | | | |
| Months | 0 | 1 | 2 | 5 | 9 | 10 | 11 | 12 | 16 |
| Patient 5 | | | | | | | | | |

| Anti-C2 | − | − | − | − | − | − | − | − | − | − | − | − | − | − |
|---|---|---|---|---|---|---|---|---|---|---|---|---|---|---|
| Anti-L4 | − | − | − | − | − | − | − | − | − | − | − | − | − | − |
| Anti-L7 | − | + | + | + | + | + | + | − | + | + | + | + | + | + |
| Anti-L11 | − | − | − | − | − | − | − | − | − | + | + | + | + | + |
| Anti-L12 | − | − | − | − | − | − | − | − | − | − | − | − | − | + |
| HCC dx (*) | | | | | | | * | | | | | | | |
| Months | 0 | 2 | 4 | 6 | 9 | 12 | 13 | 14 | 15 | 16 | 17 | 19 | 20 | 22 |
| Patient 6 | | | | | | | | | | | | | | |

| Anti-C2 | + | − | + | + | + | + |
|---|---|---|---|---|---|---|
| Anti-L4 | − | − | − | − | − | − |
| Anti-L7 | − | − | − | + | + | + |
| Anti-L11 | + | + | + | + | + | + |
| Anti-L12 | − | − | − | − | − | + |
| HCC dx (*) | | | | | | |
| Months | 0 | 1 | 2 | 5 | 10 | 15 |
| Patient 7 | | | | | | |
| Anti-C2 | − | − | + | + | + | + |
| Anti-L4 | − | − | − | − | − | − |
| Anti-L7 | − | − | − | − | − | − |
| Anti-L11 | − | − | + | + | − | + |
| Anti-L12 | + | + | + | + | − | + |
| HCC dx (*) | | | | | | |
| Months | 0 | 1 | 2 | 4 | 6 | 10 |
| Patient 8 | | | | | | |

| Anti-C2 | + | + | + | + | − | + | + | + | + | + | + |
|---|---|---|---|---|---|---|---|---|---|---|---|
| Anti-L4 | − | − | − | − | − | − | − | − | − | − | − |
| Anti-L7 | − | − | − | − | − | − | − | − | − | − | − |
| Anti-L11 | + | + | + | + | − | + | + | + | + | + | + |
| Anti-L12 | − | − | − | − | − | − | − | − | − | − | − |
| HCC dx (*) | | | | | | | | | | | |
| Months | 0 | 1 | 2 | 4 | 5 | 6 | 8 | 9 | 11 | 13 | 18 |
| Patient 9 | | | | | | | | | | | |

| Anti-C2 | − | + | + | + | + | + |
|---|---|---|---|---|---|---|
| Anti-L4 | − | − | − | − | − | − |
| Anti-L7 | − | − | − | − | − | − |
| Anti-L11 | + | + | + | + | + | + |
| Anti-L12 | − | − | − | − | − | − |
| HCC dx (*) | | * | | | | |
| Months | 0 | 3 | 5 | 6 | 8 | 10 |
| Patient 10 | | | | | | |

| Anti-C2 | + | + | + | + | + | + | + | + | + | + |
|---|---|---|---|---|---|---|---|---|---|---|
| Anti-L4 | + | + | + | + | + | + | + | + | + | + |
| Anti-L7 | − | − | − | − | − | − | − | − | − | + |
| Anti-L11 | − | + | + | + | + | + | + | + | + | + |
| Anti-L12 | − | − | − | − | − | − | − | − | − | − |
| HCC dx (*) | | | | | | | | | | * |
| Months | 0 | 1 | 3 | 6 | 10 | 14 | 15 | 16 | 26 | 45 |
| Patient 11 | | | | | | | | | | |

| | | | | | | | | | | | | | | | | | | |
|---|---|---|---|---|---|---|---|---|---|---|---|---|---|---|---|---|---|---|
| Anti-C2 | + | + | + | + | + | + | + | + | + | + | + | + | + | + | + | + | + | + |
| Anti-L4 | − | − | − | − | − | − | − | − | − | − | − | − | − | − | − | − | − | − |
| Anti-L7 | − | − | − | − | − | − | − | − | − | − | − | − | − | − | − | − | − | − |
| Anti-L11 | − | − | − | − | − | − | − | − | − | − | − | − | − | − | − | + | + | + |
| Anti-L12 | − | − | − | − | − | − | − | − | − | − | − | − | − | − | − | − | − | − |
| HCC dx (*) | | | | | | | * | | | | | | | | | | | |
| Mos. | 0 | 4 | 7 | 12 | 14 | 17 | 18 | 21 | 22 | 24 | 25 | 27 | 28 | 29 | 30 | 31 | 32 | 34 |

All references cited with respect to synthetic, preparative and analytical procedures are incorporated herein by reference. All sequence records identified by GenBank accession numbers are incorporated herein by reference.

The present invention may be embodied in other specific forms without departing from the spirit or essential attributes thereof and, accordingly, reference should be made to the appended claims, rather than to the foregoing specification, as indication the scope of the invention.

What is claimed is:

1. A method for identifying individuals at risk for hepatocellular carcinoma (HCC) comprising:
testing a blood sample for one or more marker proteins in the sample, or for one or more marker antibodies which bind to said one or more marker proteins, which marker protein is the product of a cellular gene which is differentially expressed in HBxAg[+] cells as compared with HBxAg[−] cells, the presence of said one or more marker proteins or marker antibodies in the sample being indicative of a risk for developing hepatocellular carcinoma, said one or more marker proteins encoded by nucleic acids selected from the group consisting of SEQ ID NO:1, SEQ ID NO:2, SEQ ID NO:3, SEQ ID NO:4, SEQ ID NO:5, and SEQ ID NO:9.

2. The method according to claim 1, wherein the one or more marker proteins are encoded by nucleic acids selected from the group consisting of SEQ ID NO:1 and SEQ ID NO:9.

3. The method of claim 1, wherein the blood sample is tested for one or more of said marker proteins in the sample by immunoassay, comprising contacting the sample with one or more antibodies which bind said marker proteins.

4. The method according to claim 1, wherein the immunoassay is a radioimmunoassay, an immunoufluorescence assay, a chemiluminescence assay or an enzyme-linked immunosorbent assay.

5. The method according to claim 4, wherein the immunoassay is an enzyme-linked immunosorbent assay.

6. A method for identifying individuals at risk for HCC comprising:
testing a blood sample for one or more marker proteins in the sample, or for one or more marker antibodies which bind to said one or more marker proteins, which marker protein is the product of a cellular gene which is differentially expressed in HBxAg[+] cells as compared with HBxAg[−] cells, the presence of said one or more marker proteins or marker antibodies in the sample being indicative of a risk for developing hepatocellular carcinoma, said one or more marker proteins encoded by nucleic acids selected from the group consisting of SEQ ID NO:1, SEQ ID NO:2, SEQ ID NO:3, SEQ ID NO:4, SEQ ID NO:5, and SEQ ID NO:9, further wherein the one or more antibodies bind to an antigen of one or more marker proteins encoded by nucleic acids selected from the group consisting of SEQ II) NO:1, SEQ ID NO:2, SEQ ID NO:3, SEQ ID NO:4, SEQ ID NO:5, and SEQ ID NO:9.

7. The method according to claim 6 wherein the antibody binds to an antigen of a marker protein selected from the group consisting of SEQ ID NO:1 and SEQ ID NO:9.

8. The method according to claim 6 wherein the blood sample is tested for said one or more antibodies by an immunoassay selected from the group consisting of a radioimmunoassay, an immunofluorescence assay, a chemiluminescence assay and an enzyme-linked immunosorbent assay.

9. The method according to claim 8 wherein the immunoassay is an enzyme-linked immunosorbent assay.

10. The method according to claim 9 wherein the blood sample is contacted with an immunoreagent comprising one or more peptides selected from the group consisting of SEQ ID NO:16, SEQ ID NO:17, SEQ ID NO:20, SEQ ID NO:21, SEQ ID NO:22, SEQ II) NO:23, and SEQ ID NO:24.

11. A method for identifying individuals at risk for HCC comprising: testing a blood sample for one or more marker antibodies which bind to said one or more marker proteins, which marker protein is the product of a cellular gene which is differentially expressed in HBxAg[+] cells as compared with HBxAg[−] cells, the presence of said one or more marker antibodies in the sample being indicative of a risk for developing hepatocellular carcinoma, wherein the blood sample is contacted with an immunoreagent comprising one or more peptides selected from the group consisting of SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, and SEQ ID NO:26.
